(19) United States
(12) Patent Application Publication
(10) Pub. No.: US 2025/0213466 A1
(43) Pub. Date: Jul. 3, 2025

(54) Intra-Incisional Injection of Antibiotics and Other Injections Into Skin and Mucosa

(71) Applicant: Dowling Stough, Hot Springs, AR (US)

(72) Inventor: Dowling Stough, Hot Springs, AR (US)

(21) Appl. No.: 19/002,844

(22) Filed: Dec. 27, 2024

Related U.S. Application Data
(60) Provisional application No. 63/572,336, filed on Mar. 31, 2024.
(60) Provisional application No. 63/615,137, filed on Dec. 27, 2023.

(51) Int. Cl. A61K 9/00; A61J 1/20; A61K 31/546

(52) U.S. Cl. CPC A61K 9/0019; A61J 1/2096; A61K 31/546

(57) ABSTRACT

A method for injecting a therapeutic agent into a patient's skin or mucosa. The method uses a vial containing a therapeutic agent and a syringe containing a diluent comprising an aqueous solution. Attach a closed system drug transfer (CSDT) device to the syringe. Attach the CSDT device to the vial. Transfer the diluent from the syringe into the vial through the CSDT. Mix the diluent and the therapeutic agent to make a therapeutic agent solution mixture. Fill the syringe with the therapeutic agent solution mixture. The concentration of the therapeutic agent in the therapeutic agent solution mixture is ≤5 mg/ml. Inject the therapeutic agent solution mixture into skin or mucosa of the patient. Also disclosed are other injection methods and injection kits.

### TECHNICAL FIELD

This invention relates to injections for localized drug delivery, such as for antibiotic prophylaxis against surgical site infections.

### BACKGROUND

Despite many advances in surgical procedures and technology, surgical site infections (SSI) still remain a significant concern. SSIs are a common complication of surgery. SSIs contribute to prolonged hospital stays, increased healthcare costs, and in severe cases, can even lead to mortality. Using prophylactic oral or intravenous antibiotics has been a cornerstone in preventing SSIs. However, they are often insufficiently effective, especially in high-risk surgeries or with antibiotic-resistant bacterial strains.

There is long-standing interest in the development of more effective approaches to mitigate the risk of SSIs. One promising approach is using intra-incisional antibiotics—a technique involving the localized delivery of antibiotics directly into the surgical incision site. This technique offers potential advantages over systemically administered antibiotics (e.g. oral or intravenous), including the following: localized drug delivery directly to the target site at high concentration; reduced systemic side effects; reduce the risk of antibiotic resistance; avoid the nausea that often accompanies oral antibiotics; avoid uncertainty about the timing of administration prior to surgery; avoid altering the beneficial normal gut microbiome; and reduce the incidence of Clostridium difficile infection.

Despite the promising potential of intra-incisional antibiotics, there remains a need for further advancements in formulation design, delivery systems, and efficacy assessment. In particular, administering intra-incisional antibiotics is inconvenient because current antibiotic formulations are highly concentrated and require significant dilution to achieve the correct concentration for intra-incisional injection. Otherwise, local high concentrations of antibiotic can cause sloughing of skin.

Moreover, the mixing and dilution tasks are complicated, and therefore should be performed by skilled medical personnel (such as a registered nurse or pharmacist). Solving these challenges could promote widespread adoption of intra-incisional antibiotics into clinical practice, thereby ultimately improving patient outcomes and reducing the burden on healthcare systems.

### SUMMARY

In one aspect, this invention uses a closed system drug transfer (CSDT) device. Examples of such include vial adapters, adapter caps, multi-chambered syringes (e.g. having dual chambers), syringe adapters (e.g. providing an assembly with dual chambers), syringe cartridges, staked needle syringes, vial mixers, etc. Specific product examples of CSDT devices include Phaseal (by BD), SmartSite (by BD), ChemoClave (by ICU Medical), ChemoLock (by ICU Medical), Equashield, and Companion (by Credence).

This invention further uses a liquid diluent, which comprises an aqueous solution. Examples of aqueous solutions include plain water, saline, lactated Ringer's solution, 5% dextrose in water, etc. In some embodiments, the liquid diluent further comprises a local anesthetic agent. Examples of such include lidocaine, benzocaine, and bupivacaine. In some embodiments, the liquid diluent further comprises a buffering agent such as sodium bicarbonate.

This invention further uses one or more therapeutic agents, which could be any substance (including small molecule drugs and biologics) conventionally used in medical treatment such as anti-microbial drugs (e.g. antibiotics, antivirals, or antifungals), anti-inflammatory drugs (e.g. corticosteroids), anti-neoplastic drugs, anti-androgen drugs, or cardiovascular drugs. The therapeutic agent could be in liquid or powder form (e.g. lyophilized). As used herein, “therapeutic agent solution mixture” means a mixture comprising a diluent and a therapeutic agent, and optionally other ingredients such as a buffering agent, a local anesthetic, epinephrine, etc.

Use any suitable amount of the therapeutic agent. In the context of the therapeutic agent being in powder form and contained within a vial or the CSDT device (such as within one of the chambers of a dual chamber syringe), such could contain ≤100 mg of the powder therapeutic agent; and in some cases, ≤50 mg. To define a lower limit, such could contain ≥1 mg of the powder therapeutic agent. In one embodiment, the therapeutic agent is ceftriaxone in powder form contained in a vial. In some embodiments, the amount of ceftriaxone contained in the vial is ≤300 mg; and in some cases, about 250 mg. In some embodiments, the amount of ceftriaxone contained in the vial is ≤100 mg; and in some cases, about 50 mg.

In the context of the therapeutic agent being in liquid form and contained within a vial or the CSDT device (such as within one of the chambers of a dual chamber syringe), such could contain ≤1.0 ml of the liquid therapeutic agent; and in some cases, ≤0.5 ml. To define a lower limit, such could contain ≥0.05 ml of the liquid therapeutic agent. These smaller dose amounts may be particularly useful in the context of targeted localized delivery of the therapeutic agent (e.g. intra-incisional or intra-lesional). Because the therapeutic agent is administered locally at the target site, such smaller dose amounts are feasible.

In another aspect, this invention is a method for injection into a patient's skin or mucosa. The injection could be performed for various purposes. In some embodiments, the injection is for treatment of a skin condition by intralesional injection. Examples of such include epidermal inclusion cyst, keloid scar, psoriasis, nodular prurigo, etc. Inject the therapeutic agent into the skin lesion. As an example, treat hypertrophic scars on the skin with intralesional injection of triamcinolone (corticosteroid) and 5-fluorouracil as a combination of two therapeutic agents.

In some embodiments, the injection is for performing intra-incisional injection of antibiotic for prophylaxis against surgical site infection. Perform the injection in preparation for making an incision for a surgical procedure. The site of the incision could be any part of the body in which surgical incisions into skin or mucosa are made, such as oral, nasal, anal, vaginal, etc. Examples of such surgical procedures include Mohs skin surgery, inguinal hernia surgery, cholecystectomy, hysterectomy, inguinal hernia repair, spinal surgery, joint surgery, tonsillectomy, hemorrhoidectomy, oral surgery, etc.

In this context, the therapeutic agent is a bacterial antibiotic drug. Examples of such include clindamycin, ceftriaxone, sarecycline, nafcillin, ofloxacin, vancomycin, gentamicin, doxycycline, trimethoprim/sulfa, daptomycin, ciprofloxacin, cephalexin, cefdinir, cefuroxime, and cefaclor. Inject the antibiotic solution mixture into a target incision site for the surgery. Make a surgical incision at the target incision site. Because the antibiotic is delivered directly into the incision site at an effective concentration, the surgical incision could be made relatively soon thereafter (e.g. within 20 minutes).

The method of this invention comprises mixing the therapeutic agent with the liquid diluent. The manner in which this is performed will vary according to various conditions, such as the type of CSTD device used, whether the therapeutic agent is in liquid or powder form, the content or composition of the liquid diluent, whether vials are used and the contents therein, etc.

In some embodiments, the therapeutic agent is contained in a vial and the liquid diluent is contained in a syringe (single chamber). The syringe may be prefilled with the liquid diluent. Alternatively, the liquid diluent may be provided in a different vial and the user draws the liquid diluent (from the vial) into the syringe. Attach the CSDT device to the syringe. Also attach the CSDT device to the vial. Perform mixing by injecting the liquid diluent (from the syringe) into the vial for the therapeutic agent through the CSDT device. This mixing of the diluent and therapeutic agent creates a therapeutic agent solution mixture. Draw the solution mixture back into the syringe. Optionally, add a local anesthetic, or a buffering agent, or both into the therapeutic agent solution mixture. Optionally, detach the CSDT device from the syringe or the vial after filling the syringe with the therapeutic agent solution mixture.

In the context of the buffering agent being included in the therapeutic agent solution mixture, the buffering agent could be added in any suitable manner. For example, the context could be a syringe that is provided prefilled with a local anesthetic, the buffering agent provided in a vial, and the therapeutic agent provided in a different vial. In this context, mixing of the three components could be performed by drawing up the buffering agent into the syringe to mix with the local anesthetic, then injecting the local anesthetic mixture into the therapeutic agent vial to create a therapeutic agent solution mixture, and then drawing up the therapeutic agent solution mixture back into the syringe.

In some embodiments, the CSDT device is a multi-chambered syringe that comprises a first chamber and a second chamber (and optionally, additional chambers). The first chamber contains a liquid diluent and the second chamber contains a therapeutic agent. The mixing process comprises pushing the plunger of the syringe to cause the liquid diluent to flow into the second chamber (containing the therapeutic agent), or to cause the therapeutic agent (if in liquid form) to flow into the first chamber (containing the liquid diluent). This mixing process creates a solution mixture of the therapeutic agent. The second chamber (containing the therapeutic agent) could be located proximal to the first chamber (containing the diluent), or vice versa. In some cases, the therapeutic agent solution mixture is created in the proximally-located chamber.

The final concentration of the therapeutic agent solution mixture in the syringe (whether single or multi-chamber) for injection into the patient could be ≤5 mg/ml; and in some cases, ≤2.5 mg/ml; and in some cases, ≤1.0 mg/ml. To define a lower limit, the final concentration of the therapeutic agent could be ≥0.1 mg/ml. In the context of ceftriaxone (as the therapeutic agent), the final concentration of ceftriaxone in the therapeutic agent solution mixture could be ≤75 mg/ml; and in some cases, ≤35 mg/ml; and in some cases, about 25 mg/ml.

In some embodiments, the method involves using two or more CSDT devices in sequence. The CSDT devices may be the same type or different types. For example, both the first and second CSDT devices could be an identical pair of vial adaptors. For another example, the first CSDT device could be a dual-chambered syringe and the second CSDT could be a vial adaptor. In this example, the dual-chambered syringe could contain a local anesthetic in one chamber and a therapeutic agent in the other chamber. In this example, the kit could further comprise a vial containing a buffering agent. In some cases, after mixing ingredients with the first CSDT device, detach the first CSDT device from the syringe, attach the second CSDT device, and repeat the same mixing procedure with a different vial containing a different ingredient (e.g. a buffering agent). In some cases, the first CSDT device is a multi-chamber syringe and after mixing the ingredients in the syringe, attach the second CSDT device to the syringe and perform mixing with a vial containing a different ingredient.

In another aspect, this invention is an injection kit comprising a CSDT device and a therapeutic agent. The components of the kit will vary according to various conditions, such as the type of CSTD device that is used, whether the therapeutic agent is in liquid or powder form, the content or composition of the liquid diluent, whether vials are used and the contents therein, etc. In this injection kit, the components are provided together in the same package.

In some embodiments, the kit further comprises a vial containing the therapeutic agent. The kit further comprises a syringe, which is optionally prefilled with a diluent. The kit optionally comprises a second vial that contains the diluent. In some embodiments, the CSDT device is a multi-chambered syringe and the therapeutic agent is contained in a chamber of the syringe. The kit may further comprise a vial containing a buffering agent. In some embodiments, the kit further comprises a second CSDT device which may be of the same or a different type than the first CSDT device. Having multiple CSDT devices could be useful for performing a mixing procedure that involves using the CSDT device in sequence to mix different ingredients together.

In another aspect, this invention is an injection kit comprising a vial access device, a vial containing a therapeutic agent, and optionally, an intradermal injection needle. One example of a vial access device is a vented vial access needle, such as the Nokor needle (by BD). In one embodiment, the therapeutic agent is ceftriaxone in powder form. In some embodiments, the amount of ceftriaxone contained in the vial is ≤100 mg; and in some cases, about 50 mg.

In some embodiments, the injection kit further comprises another vial containing a diluent. The volume of diluent in the vial may be ≤15 ml or ≤10 ml. To define a lower limit, the volume of diluent may be ≥0.1 ml. A method of using the aforementioned kit for injecting a therapeutic agent into a patient's skin may comprise the following steps. Attach the vial access device to a syringe. Insert the vial access device into the vial containing the diluent and draw the diluent through the vial access device into the syringe. Insert the vial access device into the vial containing the therapeutic agent. Inject the diluent in the syringe into the vial containing the therapeutic agent. Mix the diluent with the therapeutic agent to form an injection mixture. Detach the vial access device from the syringe. Attach the intradermal injection needle to the syringe. Inject the injection mixture into the patient's skin. In some embodiments, the injection is made exclusively into a dermis layer of the patient's skin.

In the same manner as explained above, the injection could be for performing intra-incisional injection of antibiotic for prophylaxis against surgical site infection (in which the therapeutic agent is an antibiotic). The injection is performed in preparation for making an incision for a surgical procedure. Inject the injection mixture into a target incision site for the surgery. Make a surgical incision at the target incision site. Because the antibiotic is delivered directly into the incision site at an effective concentration, the surgical incision could be made relatively soon thereafter (e.g. within 20 minutes).

In some embodiments, instead of another vial containing the diluent, the injection kit further comprises a syringe that contains (pre-filled with) the diluent. The volume of diluent in the syringe may be ≤15 ml or ≤10 ml. To define a lower limit, the volume of diluent may be ≥0.1 ml. A method of using the aforementioned kit for injecting a therapeutic agent into a patient's skin may comprise the following steps. Attach the vial access device to the syringe (containing the diluent). Insert the vial access device into the vial containing the therapeutic agent. Inject the diluent in the syringe into the vial containing the therapeutic agent. Mix the diluent with the therapeutic agent to form an injection mixture. Detach the vial access device from the syringe. Attach the intradermal injection needle to the syringe. Inject the injection mixture into the patient's skin. In some embodiments, the injection is made exclusively into a dermis layer of the patient's skin.

In the same manner as explained above, the injection could be for performing intra-incisional injection of antibiotic for prophylaxis against surgical site infection (in which the therapeutic agent is an antibiotic). The injection is performed in preparation for making an incision for a surgical procedure. Inject the injection mixture into a target incision site for the surgery. Make a surgical incision at the target incision site. Because the antibiotic is delivered directly into the incision site at an effective concentration, the surgical incision could be made relatively soon thereafter (e.g. within 20 minutes).

### BRIEF DESCRIPTION OF THE DRAWINGS

FIGS. 1A-1H show an example method of this invention.

FIG. 2 shows an example kit of this invention.

FIG. 3A-3F show another example method this invention.

FIG. 4 shows another example kit of this invention.

FIG. 5A-5C show another example method of this invention.

FIGS. 6A-6F show another example method of this invention.

FIG. 7 shows another example kit of this invention.

FIG. 8 shows an example vial adaptor that could be used in this invention.

FIG. 9 shows an example of a vented vial access needle that could be used in this invention.

FIG. 10 shows another example kit of this invention.

FIG. 11 shows another example kit of this invention.

### DETAILED DESCRIPTION OF EXAMPLE EMBODIMENTS

Drawings are provided to help understand the invention and illustrate specific representative examples. The drawings herein are not necessarily made to scale or actual proportions. For example, the size of components may be adjusted to accommodate the page size.

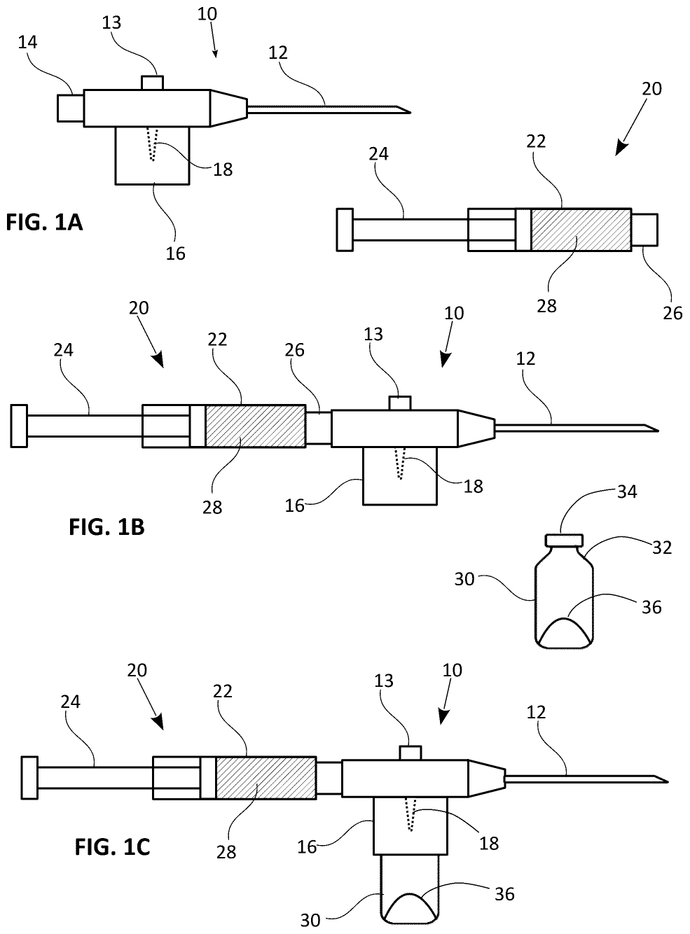

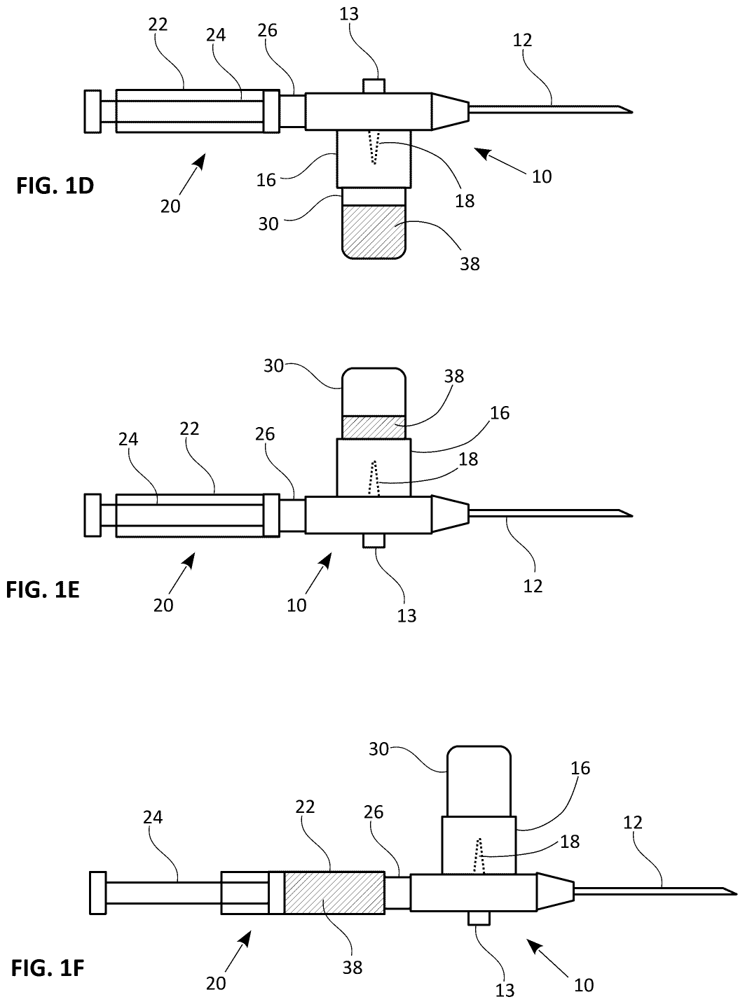

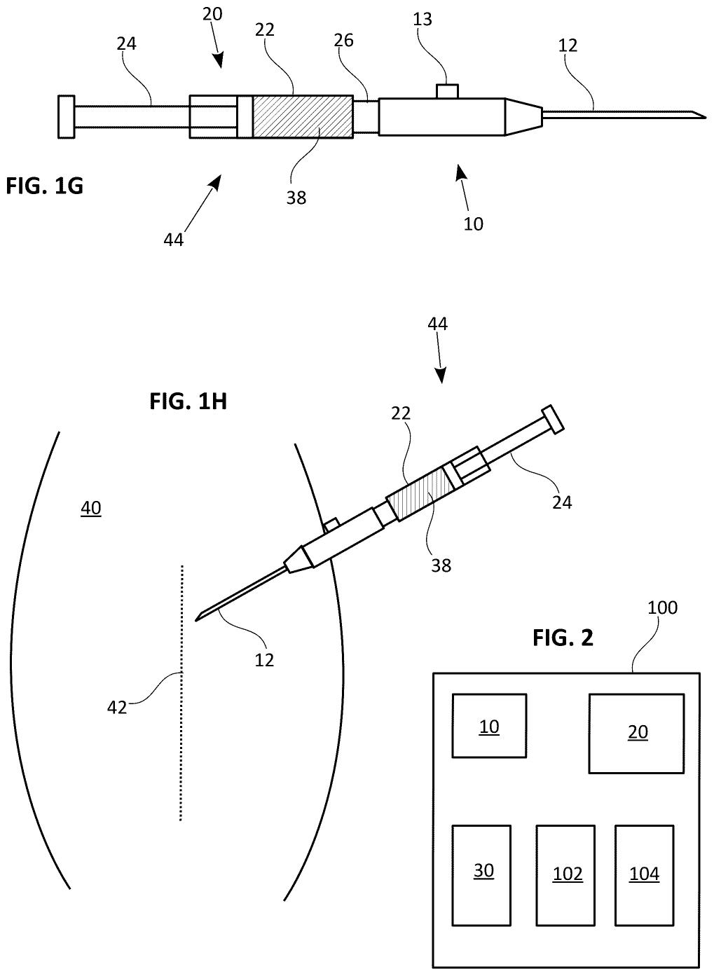

FIGS. 1A-1H show an example of this invention. FIG. 1A shows a vial adaptor 10 and a syringe 20 alongside. Vial adaptor 10 has an injection needle 12 pre-mounted thereon. On the other side, vial adaptor 10 has a Luer-type connector 14 for connecting with syringe 20. On the bottom side, vial adaptor 10 has a skirt 16 that defines a cavity therein to receive a vial. A spike 18 protrudes inside skirt 16.

Syringe 20 is a conventional medication syringe that comprises a barrel 22, plunger 24, and a Luer-type connector 26. In the example of FIG. 1A, a lidocaine solution 28 has already been drawn up into syringe 20 (from a separately provided vial of lidocaine solution, not shown here). FIG. 1B shows syringe 20 (containing lidocaine solution 28) securely connected to vial adaptor 10 via Luer connector 26. Alongside, FIG. 1B further shows an antibiotic vial 30 containing an antibiotic 36 provided as lyophilized powder. Antibiotic vial 30 has a neck 32 and cap 34. There is a seal (not shown in this side view) at the top of cap 34. Antibiotic 36 must be reconstituted in an aqueous solution in order to be used.

In FIG. 1C, antibiotic vial 30 is placed alongside vial adaptor 10. Neck 32 of vial 30 is positioned inside skirt 16 of vial adaptor 10 such that the seal on cap 34 is under spike 18. Vial adaptor 10 is pressed down against antibiotic vial 30 so that spike 18 punctures through the seal on cap 34. Vial adaptor 10 is further advanced against vial 30 until neck 32 is completely fitted inside skirt 16 with spike 18 inside of vial 30.

In FIG. 1D, antibiotic 36 in vial 30 is reconstituted by lidocaine aqueous solution 28 contained within syringe 20. To reconstitute antibiotic 36, the user pushes plunger 24 on syringe 20 forward. This drives lidocaine solution 28 out of syringe 20, into vial adapter 10, and out through a side hole (not shown) on spike 18. This fills vial 30 with lidocaine solution 28 that dissolves antibiotic 36. The user swirls or shakes vial 30 (together with vial adapter 10) to promote dissolution of antibiotic 36 and to result in reconstituted antibiotic solution 38.

In FIG. 1E, the user performs filling of syringe 20 with antibiotic solution 38. The user inverts vial adapter 10 so that skirt 18 and attached vial 30 point upwards. In FIG. 1F, the user pulls back on plunger 24 to withdraw antibiotic solution 38 out of vial 30 through the side hole in spike 18 and draw it into syringe 20. The user inverts again vial adaptor 10 and presses button 13 to eject skirt 16 and attached vial 30. In FIG. 1G, the assembly 44 of syringe 20, vial adapter 10 (portion remaining after skirt 16 is detached), and injection needle 12 is now ready to inject antibiotic solution 38 into the patient.

FIG. 1H shows a surgical area 40 with a target site 42 indicated where the surgical incision is planned. Before the incision is made, antibiotic solution 38 is injected into the skin and subcutaneous tissue along target site 42 via intradermal needle 12 on the device assembly. The shaft of intradermal needle 12 has a length of 2-15 mm and a diameter of 24-32 gauge. After the antibiotic prophylaxis, the surgeon makes the incision at target site 42 and performs the surgical procedure.

FIG. 2 shows an example of a kit 100 for vial adaptor 10 as a block diagram. Kit 100 contains the following items: vial adaptor 10, empty syringe 20, antibiotic vial 30, vial 102 of lidocaine solution, and vial 104 of bicarbonate solution. The bicarbonate solution could be added to buffer the acidic lidocaine and reduces the burning pain sensation during skin infiltration.

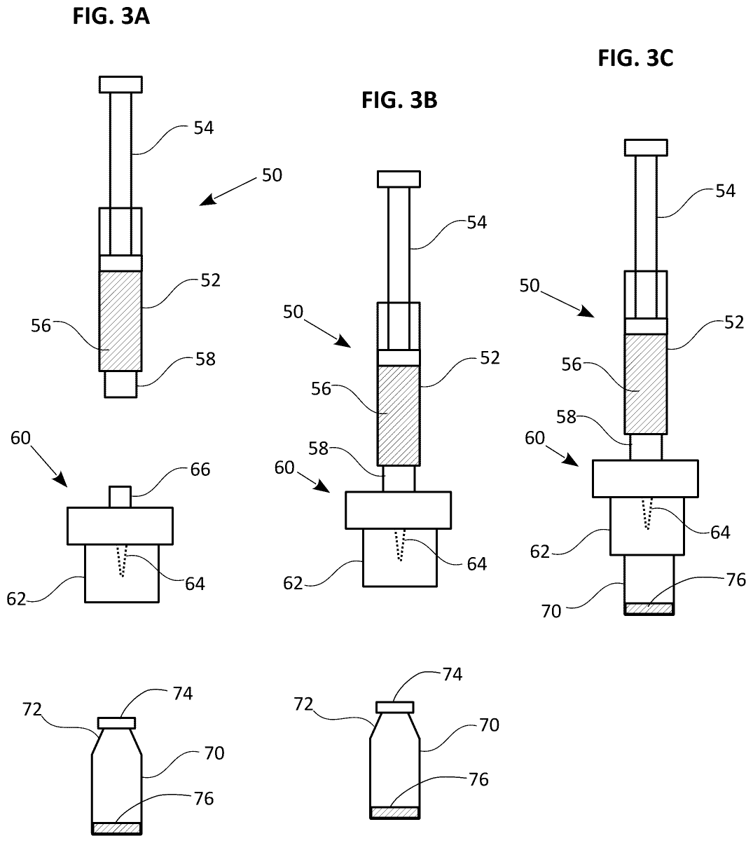

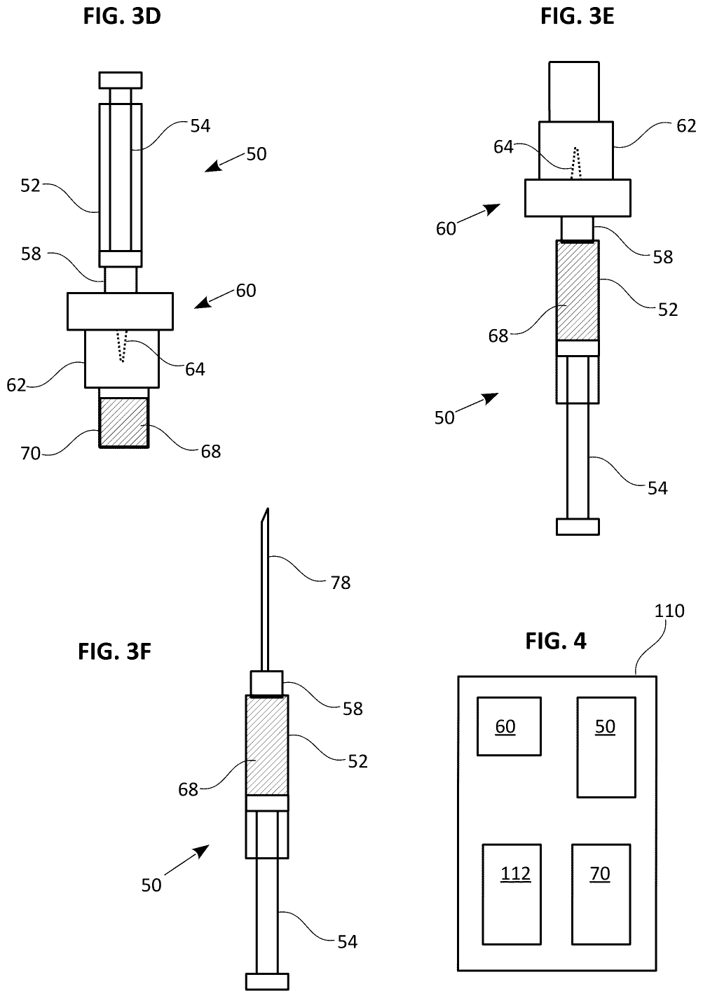

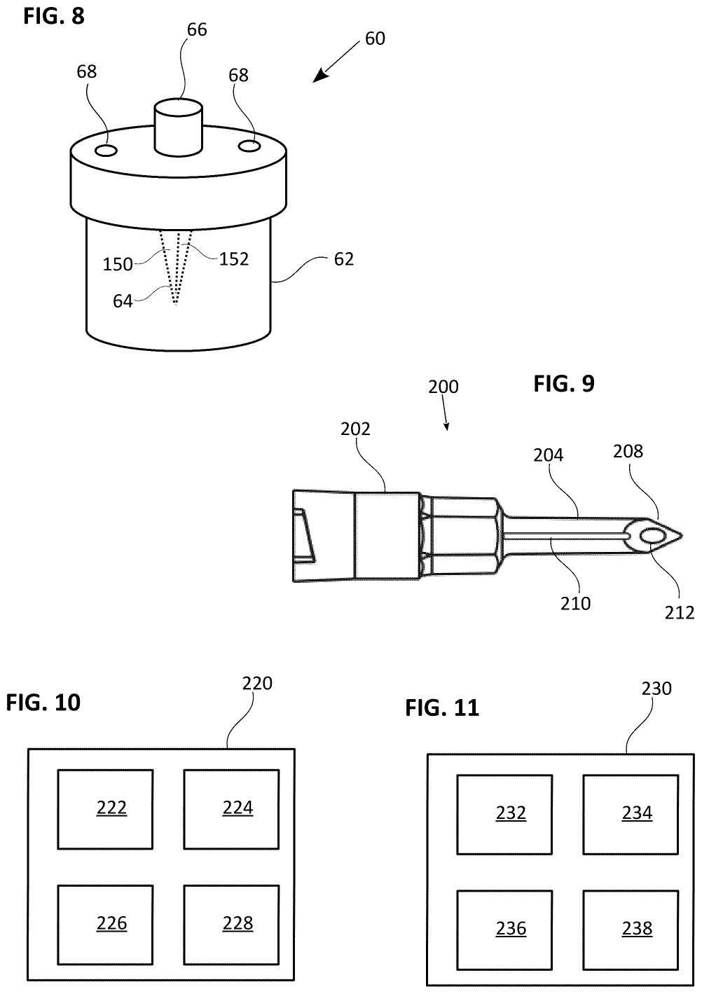

FIG. 3A-3F show another example of this invention. FIG. 3A shows a conventional syringe 50 that is provided prefilled with a lidocaine solution 56. Syringe 50 comprises a barrel 52, plunger 54, and Luer-type connector 58. Further shown is a vial adaptor 60 that comprises a syringe connector 66, a skirt 62, and a spike 64 in the interior of skirt 62. FIG. 8 shows an isolated view of vial adaptor 60 with explanation thereof below. Further shown is a vial 70 containing a small volume of antibiotic solution 76. Vial 70 comprises a neck 72 and cap 74 with a seal (not shown).

FIG. 3B shows vial adaptor 60 attached (via its syringe connector 66) to the distal end of syringe 50 via its Luer connector 58. In FIG. 3C, vial 70 is positioned under skirt 62 of vial adaptor 60. Syringe 50 is pressed down to push vial adaptor 60 against vial 70. Spike 64 punctures through the seal on cap 74 of vial 70. By continuing to press down, vial adaptor 60 is advanced against vial 70 until neck 72 is completely fitted inside skirt 62 with spike 64 inside of vial 70.

In FIG. 3D, plunger 54 of syringe 50 is pushed down to inject lidocaine solution 56 into vial 70. This mixes with antibiotic solution 76 to create mixture solution 68. As lidocaine solution 56 is injected into vial 70, displaced air inside vial 70 escapes through the air holes (not shown here) in vial adaptor 60. As shown in FIG. 3E, this assembly is inverted so that mixture solution 68 is drawn back into syringe 50 by pulling back on plunger 54. FIG. 3F shows syringe 50 filled with mixture solution 68 and an injection needle 78 attached thereon. Syringe 50 is now ready for use to inject mixture solution 68 into a patient.

FIG. 4 shows an example of a kit 110 for vial adaptor 60 as a block diagram. Kit 110 comprises the following items: vial adaptor 60, syringe 50 (prefilled with lidocaine), antibiotic vial 70, and a vial of bicarbonate 112 to also add to syringe 50.

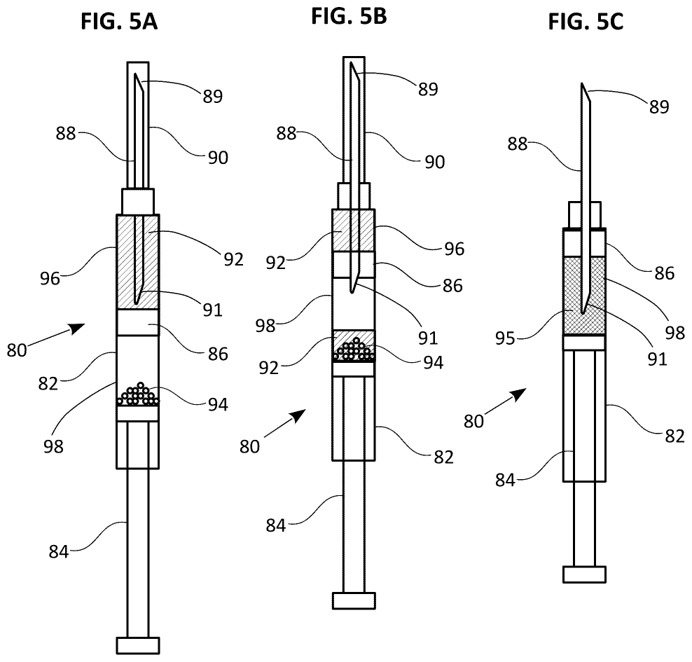

FIG. 5A-5C show another example of this invention. FIG. 5A shows a dual chamber syringe 80 that has a proximally-located chamber 98 and distally-located chamber 96. Distal chamber 96 is filled with a lidocaine solution 92. Proximal chamber 98 contains lyophilized powder antibiotic 94. The two chambers are separated by a sliding barrier 86, which has a membrane (not shown) for puncture. At the distal end of syringe 80, there is a double-pointed needle 88 that extends from inside of distal chamber 96 to outside of syringe 80.

Double-pointed needle 88 has a proximally-located internal sharpened tip 91 (inside chamber 96) and distally-located external sharpened tip 89. The distal (external) segment of needle 88 is capped with a needle sheath 90 so that fluid is not expelled during the mixing process. Syringe 80 further comprises a barrel 82 and plunger 84 that slides within barrel 82.

In FIG. 5B, antibiotic 94 is mixed with lidocaine solution 92. The user pushes forward on plunger 84. This applies pressure inside proximal chamber 98, which causes barrier 86 to slide forward in barrel 82. As this happens, internal sharpened tip 91 of needle 88 punctures through the membrane in barrier 86. Fluid pressure inside distal chamber 96 causes lidocaine solution 92 to flow into proximal chamber 98, which mixes with antibiotic 94. Lidocaine solution 92 transfers into proximal chamber 98 by flowing into a side hole (not shown) in the side of needle 88 and flowing out through internal tip 91 that has punctured through the membrane of barrier 86 and is now positioned inside proximal chamber 98.

The user swirls lidocaine solution 92 inside proximal chamber 98 to help dissolve antibiotic 94. This creates antibiotic solution 95. In FIG. 5C, the user continues to push plunger 84 forward to drive lidocaine solution 92 into proximal chamber 98 until it is filled with antibiotic solution 95. Also, sheath 90 is removed from needle 88 to expel any air remaining in proximal chamber 98. Syringe 80 is now ready for use to inject antibiotic solution 95 into a patient. Alternately, a bicarbonate solution could be further drawn into syringe 80 to buffer the lidocaine.

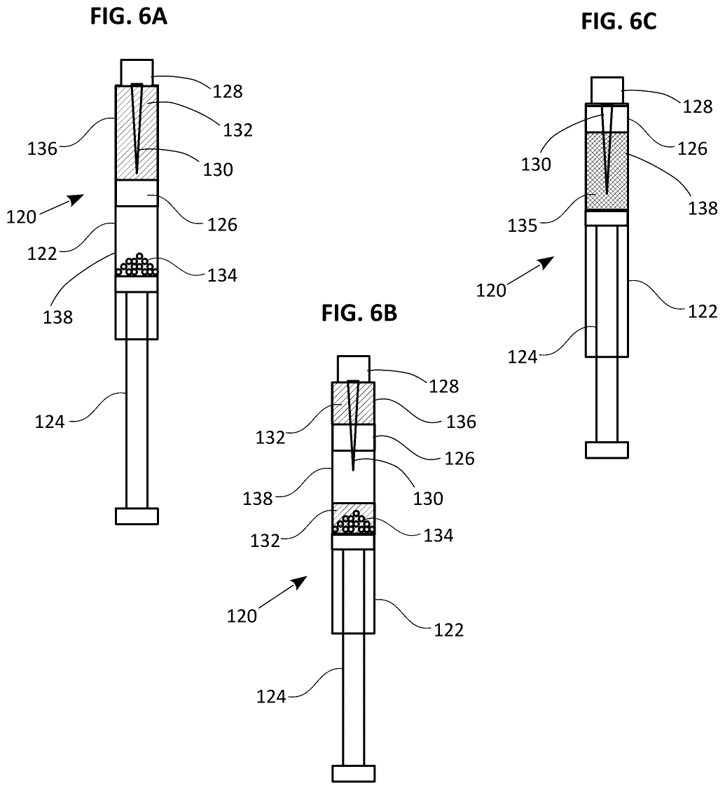

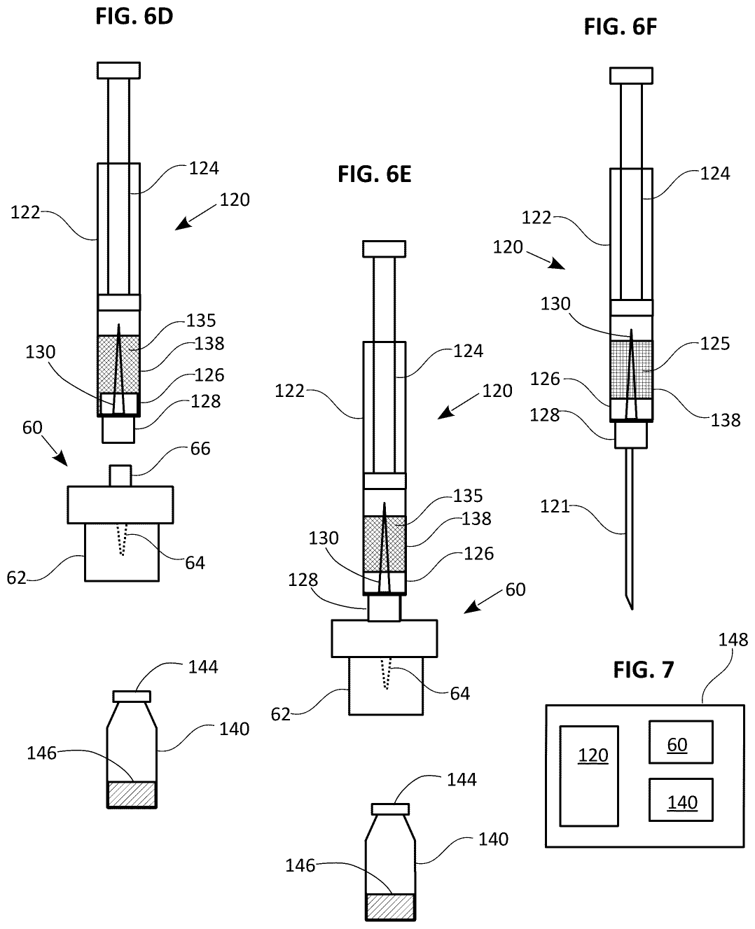

FIGS. 6A-6F show another example of this invention. FIG. 6A shows a dual chamber syringe 120 that has a proximally-located chamber 138 and distally-located chamber 136. Distal chamber 136 is filled with a lidocaine solution 132. Proximal chamber 138 contains a corticosteroid drug 134. The two chambers are separated by a sliding barrier 126, which has a membrane (not shown) for puncture. At the distal end of syringe 120, there is a stake needle 130 inside distal chamber 136 that points inward. Syringe 120 further comprises a barrel 122 and plunger 124 that slides within barrel 122.

In FIG. 6B, corticosteroid 134 is mixed with lidocaine solution 132. This is performed in the same manner as FIG. 5B above. Lidocaine solution 132 transfers into proximal chamber 138 by flowing into a side hole (not shown) in the side of needle 130 and flowing out through its sharp tip that has punctured through the membrane of barrier 126 and is now positioned inside proximal chamber 138. Lidocaine solution 132 mixes and dissolves corticosteroid 134. As shown in FIG. 6C, this creates corticosteroid solution 135 in the same manner as FIG. 5C as above.

In FIG. 6D, syringe 120 (filled with corticosteroid solution 135) is further prepared by adding bicarbonate 146 contained in vial 140 (having cap 144). This adding and mixing is performed using vial adaptor 60. As shown in FIG. 6E, the technique of using vial adaptor 60 is the same as shown in FIGS. 3A-3E above. As shown in FIG. 6F, this results in syringe 120 filled with final mixture solution 125 that contains the corticosteroid, lidocaine, and bicarbonate. Attach an injection needle 121 to Luer-type connector 128 and syringe 120 is now ready to inject mixture solution 125 into a patient.

FIG. 7 shows another example of a kit 148 as a block diagram. Kit 148 contains dual chamber syringe 120 that is prefilled with the corticosteroid drug and lidocaine solution in the separate compartments. Kit 148 further contains vial adaptor 60 and vial 140 of bicarbonate. Kit 148 can be used to perform the method shown in FIGS. 6A-6F above.

The CSDT device used in this invention could be vented or nonvented. FIG. 8 shows a perspective view of vial adaptor 60 as an example of a vented vial adaptor. Vial adaptor 60 comprises a skirt 62, a dual lumen spike 64, and syringe connector 66. As the content of the syringe is injected into the vial, the air pressure inside the vial rises. This increase in air pressure impedes the fluid injection process. That is why vial adaptor 60 further comprises air holes 68 to allow displaced air to escape the vial. Dual lumen spike 64 has a fluid lumen 150 through which injected fluid from the syringe travels. Dual lumen spike 64 further has an air lumen 152 through which displaced air exits. Air holes 68 are in communication with air lumen 152 to provide a path for displaced air to exit. Another example of a vented vial adaptor that could be used in this invention is described in U.S. Pat. No. 8,753,325 (by Nimrod Lev et al.), which is incorporated by reference herein.

FIG. 9 shows an example of a vented vial access needle 200 that could be used in this invention. Vented vial access needle 200 comprises a hub 202 and a shaft 204. The distal tip of shaft 204 has a bevel 208 and the needle lumen 212 runs through shaft 204. A small vent channel 210 on the exterior of shaft 204 extends from bevel 208 to hub 202. This vent channel 210 relieves any back pressure in the vial during injection of fluids into the vial.

FIG. 10 shows another example injection kit 220 of this invention as a block diagram. Kit 220 comprises the following items: a vented vial access needle 222, a vial 224 containing a therapeutic agent, a vial 226 containing a diluent, and an intradermal injection needle 228. In this injection kit 220, the aforementioned components are provided together in the same package.

FIG. 11 shows another example injection kit 230 of this invention as a block diagram. Kit 230 comprises the following items: a vented vial access needle 232, a vial 234 containing a therapeutic agent, a syringe 236 containing a diluent, and an intradermal injection needle 238. In this injection kit 230, the aforementioned components are provided together in the same package.

The foregoing description and examples merely illustrate the invention and are not intended to be limiting. Each of the disclosed aspects and embodiments of the invention may be considered individually or in combination with other aspects, embodiments, and variations of the invention. Also, unless otherwise specified, the steps of the methods of the invention are not limited to any particular order of performance. Persons skilled in the art may perceive modifications to these embodiments that incorporate the spirit and substance of the invention. Such modifications are within the scope of the invention.

Any use of the word “or” herein is intended to be inclusive and is equivalent to the expression “and/or,” unless the context clearly indicates otherwise. As such, for example, the expression “A or B” means A, or B, or both A and B. Similarly, for example, the expression “A, B, or C” means A, or B, or C, or any combination thereof.

1. A method for injecting a therapeutic agent into a patient's skin or mucosa, comprising:
having a vial containing a therapeutic agent;
having a syringe containing a diluent comprising an aqueous solution;
having a closed system drug transfer (CSDT) device;
attaching the CSDT device to the syringe;
attaching the CSDT device to the vial;
transferring the diluent from the syringe into the vial through the CSDT;
mixing the diluent and the therapeutic agent to make a therapeutic agent solution mixture;
filling the syringe with the therapeutic agent solution mixture, wherein the concentration of the therapeutic agent in the therapeutic agent solution mixture is ≤5 mg/ml;
injecting the therapeutic agent solution mixture into skin or mucosa of the patient.

2. The method of claim 1, wherein the injection is for treating a skin condition and the therapeutic agent solution mixture is injected into a lesion of the skin condition.

3. The method of claim 1, wherein the therapeutic agent is an antibiotic, wherein the injection is for prophylaxis in preparation for surgery, and wherein the antibiotic solution mixture is injected into a target incision site for the surgery.

4. The method of claim 3, wherein an incision is made at the target incision site within 20 minutes after injection of the antibiotic solution mixture.

5. The method of claim 1, wherein the therapeutic agent is in powder form and the amount of therapeutic agent in the vial is ≤100 mg.

6. The method of claim 1, wherein the therapeutic agent is in liquid form and the volume of therapeutic agent in the vial is ≤1.0 ml.

7. The method of claim 1, wherein the syringe is prefilled with the diluent, or wherein the step of having the syringe containing the diluent comprises filling the syringe with the diluent.

8. The method of claim 1, wherein the diluent further comprises a local anesthetic.

9. The method of claim 1, wherein the therapeutic agent is ceftriaxone.

10. The method of claim 9, wherein the amount of ceftriaxone contained in the vial is ≤100 mg.

11. An injection kit comprising:
a vial access device;
a first vial containing a therapeutic agent;
a second vial containing a liquid diluent;
an intradermal injection needle comprising a shaft.

12. The injection kit of claim 11, wherein the volume of liquid diluent in the second vial is ≤15 ml.

13. The injection kit of claim 11, wherein the therapeutic agent is ceftriaxone.

14. The injection kit of claim 13, wherein the amount of ceftriaxone contained in the first vial is ≤100 mg.

15. The injection kit of claim 11, wherein the shaft of the intradermal injection needle has a length of 2-15 mm.

16. The injection kit of claim 15, wherein the shaft of the intradermal injection needle has a diameter of 24-32 gauge.

17. The injection kit of claim 11, wherein the vial access device is a vented needle.

18. A method of injecting a therapeutic agent into a patient's skin, comprising:
having an injection kit of claim 11;
attaching the vial access device to a syringe;
inserting the vial access device into the second vial containing the liquid diluent;
drawing the liquid diluent through the vial access device into the syringe;
inserting the vial access device into the first vial containing the therapeutic agent;
injecting the liquid diluent in the syringe into the first vial containing the therapeutic agent;
mixing the liquid diluent with the therapeutic agent to form an injection mixture;
detaching the vial access device from the syringe;
attaching the intradermal injection needle to the syringe;
injecting the injection mixture into the patient's skin.

19. The method of claim 18, wherein the injection is made exclusively into a dermis layer of the patient's skin.

20. The method of claim 18, wherein the therapeutic agent is an antibiotic and the injection is performed for prophylaxis against surgical site infection.
